(19) United States
(12) Patent Application Publication
(10) Pub. No.: US 2025/0165778 A1
(43) Pub. Date: May 22, 2025

(54) SIGNAL DENOISING BASED ON ADAPTABLE DEEP NEURAL NETWORKS

(71) Applicants: SONY GROUP CORPORATION, TOKYO (JP); SONY CORPORATION OF AMERICA, NEW YORK, NY (US)

(72) Inventors: WANXIN XU, SAN JOSE, CA (US); KO-KAI ALBERT HUANG, SAN JOSE, CA (US)

(21) Appl. No.: 18/808,653

(22) Filed: Aug. 19, 2024

Related U.S. Application Data
(60) Provisional application No. 63/600,290, filed on Nov. 17, 2023.

(51) Int. Cl. G06N 3/08; A61B 5/00; A61B 5/243; A61B 5/346

(52) U.S. Cl. CPC G06N 3/08; A61B 5/243; A61B 5/346; A61B 5/7203; A61B 5/7257; A61B 5/7267

(57) ABSTRACT

An electronic device and a method for implementation for signal denoising based on adaptable deep neural networks. The electronic device receives training data comprising a first bio-signal and a second bio-signal that is different from the first bio-signal. The electronic device computes a weighted sum of the first bio-signal and the second bio-signal. The electronic device generates a mixed signal based on the weighted sum. The electronic device generates an output signal based on application of a denoising neural network (DNN) on the mixed signal. Further, the electronic device computes a loss based on a comparison of the output signal with the first bio-signal and trains the DNN for a number of epochs until the computed loss is below a threshold.

### CROSS-REFERENCE TO RELATED APPLICATIONS/INCORPORATION BY REFERENCE

This application claims priority to U.S. Provisional Patent Application Ser. No. 63/600,290 filed on Nov. 17, 2023, the entire content of which is hereby incorporated herein by reference.

### FIELD

Various embodiments of the disclosure relate to bio-signals. More specifically, various embodiments of the disclosure relate to an electronic device and a method for signal denoising based on adaptable deep neural networks.

### BACKGROUND

In the field of medical diagnostics, particularly cardiovascular disease diagnosis and analysis, bio-signals such as electrocardiogram (ECG) and magneto cardiogram (MCG) signals play a substantial role. These signals are typically collected from sensors attached to a patient and are analyzed to provide valuable insights into the patient's heart health. However, these bio-signals may be often corrupted by various types of noise, including motion artifacts, bad electrode contact to the skin, baseline wandering, and human activities. The process of removing noise from these bio-signals, known as denoising, is a challenging task due to the variety of noise sources and the weak nature of the signals. Traditional denoising methods often struggle to effectively remove noise without distorting the underlying signal. Furthermore, the computational efficiency of these methods can be a concern, especially in real-time scenarios where rapid signal analysis is paramount.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method for signal denoising based on adaptable deep neural networks is provided substantially as shown in, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for signal denoising based on adaptable deep neural networks, in accordance with an embodiment of the disclosure.

FIG. 2 is a block diagram that illustrates an exemplary electronic device of FIG. 1, in accordance with an embodiment of the disclosure.

FIG. 3 is a diagram that illustrates an exemplary training process of a denoising neural network, in accordance with an embodiment of the disclosure.

FIG. 4 is a diagram that illustrates an exemplary scenario of denoising a mixed signal, in accordance with an embodiment of the disclosure.

FIG. 5 is a flowchart that illustrates operations of an exemplary method for signal denoising based on adaptable deep neural networks, in accordance with an embodiment of the disclosure.

### DETAILED DESCRIPTION

The following described implementation may be found in an electronic device and method for signal denoising based on adaptable deep neural networks. Exemplary aspects of the disclosure may provide an electronic device that may receive training data comprising a first bio-signal and a second bio-signal that may be different from the first bio-signal. The electronic device may compute a weighted sum of the first bio-signal and the second bio-signal to generate a mixed signal. An output signal may be generated based on the application of a denoising neural network (DNN) on the mixed signal. The electronic device may compute a loss based on a comparison of the output signal with the first bio-signal and train the DNN for a number of epochs until the computed loss is below a threshold.

Cardiovascular disease diagnosis and analysis heavily rely on bio-signals like ECG/MCG. However, these signals are often corrupted by various noises such as baseline wandering and human activity, which can hinder their usability in real-world applications. Furthermore, the weak nature of these signals and their susceptibility to a wide variety of noise sources present challenges in their analysis. Additionally, the acquisition of large volumes of data for efficient analysis and performance improvement with deep learning-based approaches is not straightforward. The increase in the number of signal channels also increases the computational load, affecting the efficiency of real-time signal analysis.

The disclosed systems and methods address these challenges by providing an adaptable deep neural network-based approach for signal denoising. This approach may handle varying input sizes of signals and may accommodate single or multiple input channels. The design of a loss function that may analyze the frequency components of a signal as they vary over time enhances the performance of signal noise removal. Furthermore, a cost-effective solution for data augmentation with a wide coverage of signal-to-noise ratio (SNR) is provided to improve the performance of signal denoising. These features may enhance the usability of bio-signals in real-world applications such as early diagnosis of heart disease through wearable devices and biometrics for user authentication.

The disclosed approach may handle signals of varying input sizes, making it versatile and capable of processing a wide range of bio-signals. This adaptability may extend to the number of input channels as well, with the electronic device capable of accommodating both single and multiple input channels. This flexibility may allow the electronic device to be integrated into a variety of signal analysis systems, making it suitable for a wide range of applications.

One of the innovative features of this approach is the design of a loss function that can analyze the frequency components of a signal as they change over time. This time-frequency analysis enhances the performance of signal noise removal by allowing the system to identify and isolate noise components that may vary in frequency over time. This feature may be particularly useful in the context of bio-signals, which often contain noise components that fluctuate in frequency. Furthermore, the disclosed approach may provide a cost-effective solution for data augmentation. This solution may involve generation of synthetic data with a wide coverage of signal-to-noise ratio (SNR), which may be used to improve the performance of signal denoising. By training the deep neural network with this augmented data, the electronic device may learn to handle a wide range of noise levels, making it more robust and effective in real-world applications.

These features collectively enhance the usability of bio-signals in real-world applications. For instance, the improved signal quality may facilitate early diagnosis of heart disease through wearable devices. The denoised signals may provide more accurate and reliable data for heart rate monitoring, enabling early detection of irregularities that may indicate heart disease. Similarly, the denoised signals may also be used for biometrics in user authentication systems. By providing clean and reliable bio-signals, the electronic device may improve the accuracy and reliability of biometric authentication, enhancing the security of these systems.

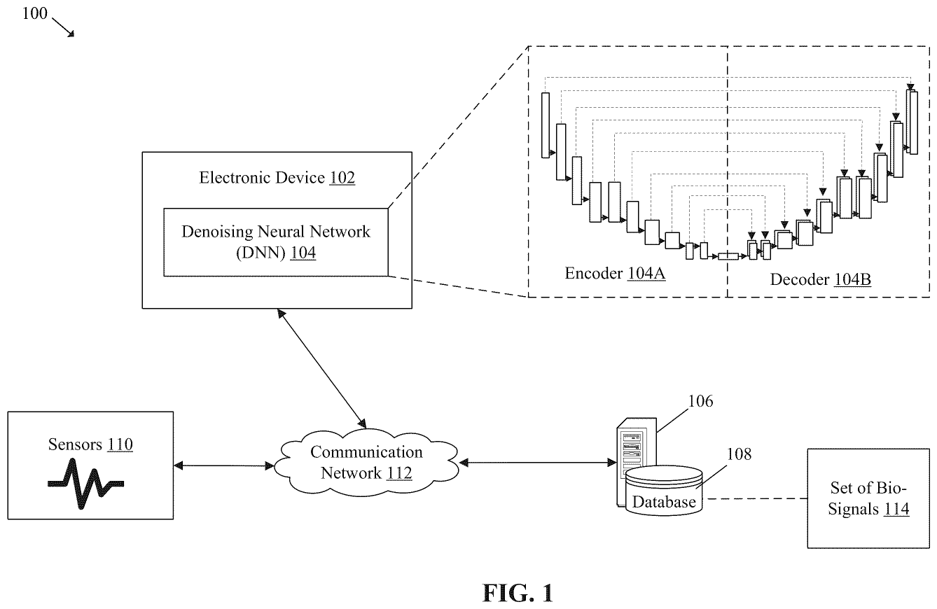

FIG. 1 is a block diagram that illustrates an exemplary network environment for signal denoising based on adaptable deep neural networks, in accordance with an embodiment of the disclosure. With reference to FIG. 1, there is shown a network environment 100. The network environment 100 may include an electronic device 102, a server 106, a database 108, sensors 110, and a communication network 112. The electronic device 102 may store a denoising neural network (DNN) 104. The sensors 110 may be associated with the electronic device 102. In FIG. 1, there is further shown a set of bio-signals 114 that may be stored on the server 106 (e.g., in the database 108) or the electronic device 102.

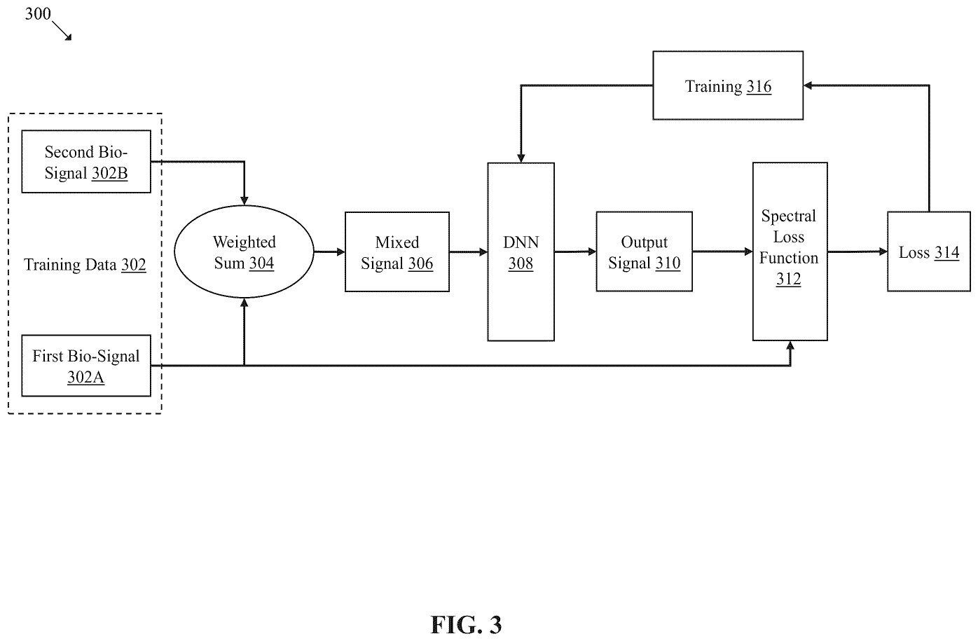

The electronic device 102 may include suitable logic, circuitry, interfaces, and/or code that may be configured to receive training data (for example, training data 302 of FIG. 3) that includes a first bio-signal (for example, a first bio-signal 302A of FIG. 3) and a second bio-signal (for example, a second bio-signal 302B of FIG. 3) that may be different from the first bio-signal. The electronic device 102 may compute a weighted sum of the first bio-signal and the second bio-signal, and may generate a mixed signal (for example, a mixed signal 306 of FIG. 3) based on the weighted sum. The electronic device 102 may generate an output signal (for example, an output signal 310 of FIG. 3) based on application of a DNN 104 on the mixed signal. The electronic device 102 may compute a loss (for example, a loss 314 of FIG. 3) based on a comparison of the output signal with the first bio-signal. Thereafter, the electronic device 102 may train the DNN 104 for a number of epochs until the computed loss is below a threshold. Examples of the electronic device 102 may include, but are not limited to, a computing device, a smartphone, a cellular phone, a mobile phone, a gaming device, a mainframe machine, a server, a computer workstation, a machine learning device (enabled with or hosting, for example, a computing resource, a memory resource, and a networking resource), a wearable device with an inbuilt bio-medical sensor, a bio-medical device, and/or a consumer electronic (CE) device.

The DNN 104 may refer to a computational framework, often based on deep learning techniques, designed to process input data through a series of layers and nodes to perform a specific task, such as denoising signals. Examples of the DNN 104 may include, but are not limited to, Convolutional Neural Networks (CNNs) for image processing, Recurrent Neural Networks (RNNs) for sequential data, a denoising autoencoder, and an encoder-decoder network (such as RPNet) which may be particularly suited for tasks like signal denoising where the input and output are signals of similar nature. In an exemplary example, the DNN 104 may be a neural network such as RP-net or a variant of RP-net that may be implemented on the electronic device 102, as discussed in, for example, Sricharan Vijayarangan, Vignesh R, Balamurali Murugesan, Preejith SP, Jayaraj Joseph, and Mohansankar Sivaprakasam, “RPnet: A Deep Learning approach for robust R Peak detection in noisy ECG” 42nd Annual International Conference of the IEEE Engineering in Medicine & Biology Society (EMBC), 2020 IEEE, 20-24 Jul. 2020, which is incorporated herein in its entirety by reference.

The DNN 104 may be a computational network or a system of artificial neurons, arranged in a plurality of layers, as nodes that may be configured to generate the output signal. The plurality of layers of the DNN 104 may include an input layer, one or more hidden layers, and an output layer. Each layer of the plurality of layers may include one or more nodes (artificial neurons, for example). Outputs of all nodes in the input layer may be coupled to at least one node of hidden layer(s). Similarly, inputs of each hidden layer may be coupled to outputs of at least one node in other layers of the DNN 104. Outputs of each hidden layer may be coupled to inputs of at least one node in other layers of the DNN 104. Node(s) in the final layer may receive inputs from at least one hidden layer to output a result. The number of layers and the number of nodes in each layer may be determined from hyper-parameters of the DNN 104. Such hyper-parameters may be set before or after training the neural network on training data.

Each node of the DNN 104 may correspond to a mathematical function (e.g., a sigmoid function or a rectified linear unit) with a set of parameters, tunable during training of the DNN 104. The set of parameters may include, for example, a weight parameter, a regularization parameter, and the like. Each node may use the mathematical function to compute an output based on one or more inputs from nodes in other layer(s) (e.g., previous layer(s)) of the DNN 104. All or some of the nodes of the neural network may correspond to the same or a different mathematical function.

In training of the DNN 104, one or more parameters of each node of the DNN 104 may be updated based on whether an output of the final layer for a given input (from the training data) matches a correct result based on a spectral loss function (for example, a spectral loss function 312 of FIG. 3) for the DNN 104. The above process may be repeated for the same or a different input until a minimum of the spectral loss function is achieved, and a training error is minimized. Several methods for training are known in art, for example, gradient descent, stochastic gradient descent, batch gradient descent, gradient boost, meta-heuristics, and the like.

The DNN 104 may include electronic data, which may be implemented as, for example, a software component of an application executable on the electronic device 102. The DNN 104 may rely on libraries, external scripts, or other logic/instructions for execution by a processing device. The DNN 104 may include code and routines configured to enable a computing device to perform one or more operations. Additionally, or alternatively, the DNN 104 may be implemented using hardware including a processor, a microprocessor (e.g., to perform or control performance of one or more operations), a field-programmable gate array (FPGA), or an application-specific integrated circuit (ASIC). Alternatively, in some embodiments, the DNN 104 may be implemented using a combination of hardware and software.

In an embodiment, the DNN 104 may be an encoder-decoder model that includes an encoder 104A and a decoder 104B. The encoder-decoder model may receive un-processed data (or a noisy signal) such as an unprocessed bio-signal as an input and may generate a denoised signal as the output signal. During training, the output signal may be compared with the first bio-signal to compute a loss using the spatial loss function. The encoder 104A may receive a mixed signal that may be based on the weighted sum of the training data as an input. Based on the received input, the encoder 104A may determine a compressed feature vector associated with the mixed signal. An encoded version (i.e., the compressed feature vector) of the received mixed signal may be input to the decoder 104B. The decoder 104B may learn to remove the second bio-signal 302B from the encoded version of the mixed signal 306 to provide the output signal 310 that includes the first bio-signal 302A. Alternatively, the decoder 104B may learn to remove the first bio-signal 302A from the encoded version of the mixed signal 306 to provide the output signal 310 that includes the second bio-signal 302B. Thus, the DNN 104 may be trained to separate the noise from the mixed signal and output a denoised signal, which is the output signal. The output signal may be a cleaner version of the mixed signal, with the noise components removed or reduced.

The electronic device 102 may be configured to input mixed signal to the encoder 104A that may generate a latent signal as an output of the encoder 104A (based on the application of the encoder 104A on input training data). As an example, the input the mixed signal may be generated based on the weighted sum of the first bio-signal and the second bio-signal that is included in the received training data. Each bio-signal may include a set of channels that define a set of attributes for the corresponding signals. The training data may be for example, a set of bio-signals such as the EEG, the ECG, the MCG, the EMG, the EOG, and the likes. Examples of the set of attributes may include, but are not limited to, width, dimensionality, a sample time, an amplitude, a frequency, and complex signal flag. In an embodiment, the DNN 104 may be deployed on the electronic device 102. In an example, the DNN 104 may be a denoising autoencoder or a variant of the denoising autoencoder. In an embodiment, the DNN 104 may be a U-net or a variant of the U-net.

The server 106 may include suitable logic, circuitry, and interfaces, and/or code that may be configured to receive training data that includes the first bio-signal and the second bio-signal. The server 106 may store the received computed weighted sum of the first bio-signal and the second bio-signal, by the electronic device 102. Further, the server 106 may store the generated mixed signal based on the weighted sum. The server 106 may store generated output signal based on application of the DNN 104 on the mixed signal. The server 106 may further store a computed loss based on a comparison of the output signal with the first bio-signal. The server 106 may provide the training data (for example the training data 302) to the DNN 104 for number of epochs until the computed loss is below the threshold.

The server 106 may be implemented as a cloud server and may execute operations through web applications, cloud applications, HTTP requests, repository operations, file transfer, and the like. Other example implementations of the server 106 may include, but are not limited to, a database server, a file server, a web server, a media server, an application server, a mainframe server, a machine learning server (enabled with or hosting, for example, a computing resource, a memory resource, and a networking resource), or a cloud computing server.

In at least one embodiment, the server 106 may be implemented as a plurality of distributed cloud-based resources by use of several technologies that are well known to those ordinarily skilled in the art. A person with ordinary skill in the art will understand that the scope of the disclosure may not be limited to the implementation of the server 106 and the electronic device 102, as two separate entities. In certain embodiments, the functionalities of the server 106 can be incorporated in its entirety or at least partially in the electronic device 102 without a departure from the scope of the disclosure. In certain embodiments, the server 106 may host the database 108. Alternatively, the server 106 may be separate from the database 108 and may be communicatively coupled to the database 108.

The database 108 may include suitable logic, interfaces, and/or code that may be configured to store the set of bio-signals 114 as timeseries data or other suitable formats. The database 108 may be derived from data off a relational or non-relational database, or a set of comma-separated values (csv) files in conventional or big-data storage. The database 108 may be stored or cached on a device, such as a server (e.g., the server 106) or the electronic device 102. The device storing the database 108 may be configured to receive a query for the bio-signals from the electronic device 102 or the server 106. For example, the query may include request for the first bio-signal and the second bio-signal. In another embodiment, the query may include request for the bio-signal received from the sensors 110. In response, the device of the database 108 may be configured to retrieve and provide the queried bio-signals to the electronic device 102 or the server 106, based on the received query.

In some embodiments, the database 108 may be hosted on a plurality of servers stored at the same or different locations. The operations of the database 108 may be executed using hardware including a processor, a microprocessor (e.g., to perform or control performance of one or more operations), a field-programmable gate array (FPGA), or an application-specific integrated circuit (ASIC). In some other instances, the database 108 may be implemented using software.

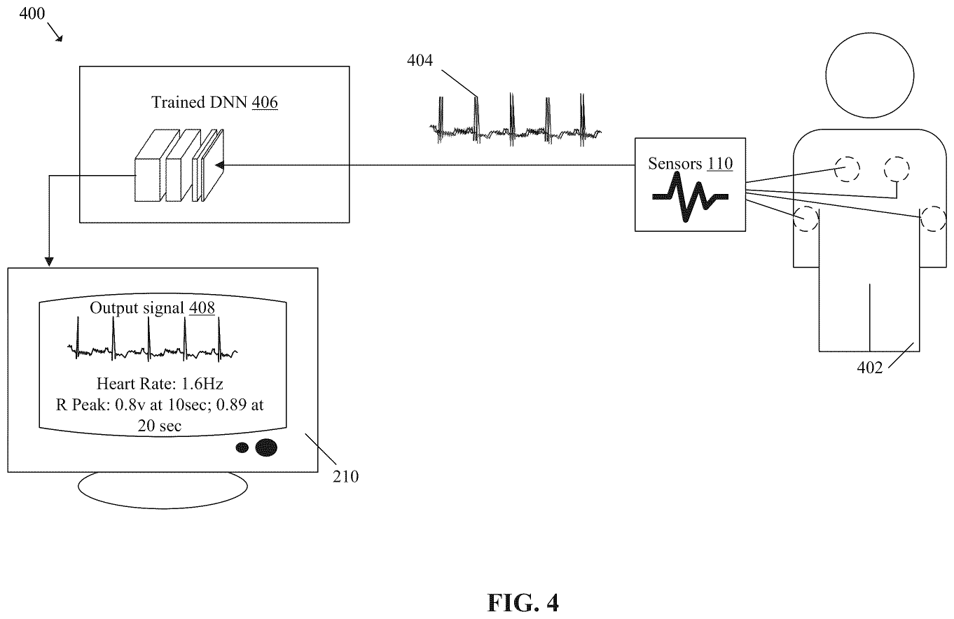

The sensors 110 may include suitable logic, circuitry, and interfaces that may be configured to capture a bio-signal of a user (for example, a user 402 of FIG. 4). In an embodiment, the sensors 110 may be an electrocardiogram (ECG) sensor. The ECG sensor may measure electrical activity of the heart around the surface of the torso of the user. For example, the ECG senor may include a set of electrodes that may be positioned on limbs and chest of the user to measure the ECG signal. In another embodiment, the sensors 110 may be a magneto cardiogram (MCG) sensor. The MCG sensor may measure an induced magnetic field of the heart at the level of the torso of the user. For example, the MCG sensor may include a superconductive quantum interference device (SQUID) that may be positioned on chest of the user to measure the MCG signal. In another embodiment, the sensors 110 may be an electroencephalogram (EEG) sensor. The EEG sensor may measure a spontaneous electrical activity of a brain of the user. For example, the EEG sensor may include a set of electrodes that may be positioned on a scalp of the user to measure the EEG. Example implementations of the sensors 110 may include, but are not limited to, a belt-type wearable sensor, a vest with embedded biosensors, a waist strap with embedded biosensors, a wrist strap with embedded biosensors, an instrumented wearable belt, a wearable garment with embedded biosensors, or a wearable article-of-manufacture having a retrofitting of biosensors.

In another embodiment, the sensors 110 may be configured to receive a touch input from the electronic device 102. The received touch input may correspond to a human touch of the user (such as the medical practitioner) on the region of a device that includes the sensors 110. The sensors 110 may be configured to measure one or more parameters associated with the user to produce the set of bio-signals 114. Based on the touch input, the sensors 110 may transmit the set of bio-signals 114 to the electronic device 102. The set of bio-signals 114 may include, for example, physiological signals and somatic sensation information associated with a defect portion (for example, a fractured bone or a diseased heart) in the portion of the body of the user.

In an embodiment, the sensors 110 may be in contact with at least the first portion of the body of the user (for example, wrist, chest, abdomen, and the likes). In another embodiment, the sensors 110 may be wrapped, wound, or strapped around the portion of the body. The sensors 110 may acquire multi-modal data through sensors, such as, but not limited to, a photoplethysmography (PPG) sensor, a temperature sensor, a blood pressure sensor, an ambient oxygen partial pressure (ppO2) sensor, or sensors which collect the somatic sensation information associated with the portion of the body. In another embodiment, the sensors 110 may be a multichannel device or a single channel device.

The set of bio-signals 114 may include data of bio-signals such as, the ECG, the MCG, the EEG, the EMG, or an electromyogram (EMG) associated with the user. The set of bio-signals 114 may be used to determine the health condition of the user.

The communication network 112 may include a communication medium through which the electronic device 102 and the server 106 may communicate with one another. The communication network 112 may be one of a wired connection or a wireless connection. Examples of the communication network 112 may include, but are not limited to, the Internet, a cloud network, Cellular or Wireless Mobile Network (such as Long-Term Evolution and 5th Generation (5G) New Radio (NR)), satellite communication system (using, for example, low earth orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 112 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 502.11, light fidelity (Li-Fi), 502.16, IEEE 502.11s, IEEE 502.11g, multi-hop communication, wireless access point (AP), device to device communication, cellular communication protocols, and Bluetooth (BT) communication protocols.

In operation, the electronic device 102 may receive the training data (such as the training data 302 of FIG. 3) comprising the set of bio-signals 114 (includes the first bio-signal and the second bio-signal that may be different from the first bio-signal). Each of the first bio-signal and the second bio-signal may be one of an electroencephalogram (EEG) signal, an electrocardiogram (ECG) signal, a magneto cardiogram (MCG) signal, an electromyogram (EMG) signal, an electrooculogram (EOG) signal, and the like. For example, if the first bio-signal is the ECG signal, then the second bio-signal may be MCG or EEG signal that is different from the first bio signal. Alternatively, if the first bio-signal is the MCG signal or the EEG signal, then the second bio-signal may be the ECG signal. The ECG signal may provide information associated with the electrical activity of the heart around the surface of the torso of the user. The MCG may provide information associated with the induced magnetic field of the heart at the level of the torso of the user. The EEG sensor provide information associated with the spontaneous electrical activity of the brain of the user. Thus, the ECG and MCG may be a graph of electric/magnetic activity of the heart respectively, versus time. The electronic device 102 may retrieve the set of bio-signals 114 from the database 108. Alternatively, the electronic device 102 may directly receive the bio-signal measured from the sensors 110. Details related to the bio-signals are further described, for example, in FIG. 3.

The electronic device 102 may compute a weighted sum of the first bio-signal and the second bio-signal. The weights for the weighted sum may be set based on a user input or a random function, providing flexibility in the generation of the mixed signal. The weighted sum may be computed based on a first weight for the first bio-signal and a second weight for the second bio-signal. In accordance with an embodiment, the second weight may be more than the first weight. In accordance with another embodiment, the second weight may be less than or equal to the first weight. For example, the first bio-signal may be represented as a1 and the first weight of a1 is w1. Further, the second bio-signal may be represented as a2 and the second weight of a2 is w2. Thus, the weighted sum may be denoted by S and may be calculated by an equation (1), as follows:

$$S = {a}_{1} \times {w}_{1} + {a}_{2} \times {w}_{2} \qquad ( 1 )$$

The electronic device 102 may generate the mixed signal based on the weighted sum. The mixed signal may be generated through a process where each sample of the mixed signal is equal to the weighted sum of a corresponding sample of the first bio-signal and a corresponding sample of the second bio-signal. Details related to the weighted sum are further provided, for example, in FIG. 3.

The mixed signal 306 may be provided as an input to the DNN 104. In an embodiment, the set of bio-signals 114 may include more bio signals apart from the first bio-signal and the second bio-signal. The other bio-signal may be different from the first bio signal. The electronic device 102 may receive the training data (such as the training data 302 of FIG. 3) comprising the set of bio-signals 114. For example, the set of bio-signals 114 may include the first bio-signal, the second bio-signal and a third bio signal. The second bio-signal and the third bio signal may be different from the first bio-signal. For example, if the first bio-signal is the ECG signal, then the second bio-signal may be MCG and the third bio-signal may be the EEG signal.

The electronic device 102 may generate an output signal based on application of the DNN 104 on the mixed signal. Details related to the output signal are further described, for example, in FIG. 3.

The electronic device 102 may compute a loss based on a comparison of the output signal with the first bio-signal. The first bio-signal may serve as a ground truth for the computation of the loss. The loss may be computed based on a spectral loss function. The spectral loss function may be a type of loss function that measures the difference between the spectral components of two signals. It is designed to capture discrepancies in the frequency domain, often using transformations such as the Short-Time Fourier Transform (STFT) or Gabor transform to convert the signals into their spectral representations. By comparing the frequency components of a predicted signal (i.e., the output signal) with those of a ground truth signal (i.e., the first bio-signal), the spectral loss function may assess the quality of signal reconstruction or denoising, emphasizing the preservation of temporal and frequency characteristics. The spectral loss function may be computed for each data point in the training data (such as, the first bio-signal and the second bio-signal), and then the backpropagation may be performed to update the parameters of the DNN 104. The spectral loss function may be used to optimize the parameters (i.e., weights) of the DNN 104 by minimizing the loss, which means that the DNN 104 makes fewer mistakes on the training data. The spectral loss function evaluates the performance of the DNN 104 and guides the optimization process by indicating the difference in the output signal from the first bio-signal. Details related to the loss are further described, for example, in FIG. 3.

The electronic device 102 may train the DNN 104 for a number of epochs until the computed loss is below a threshold. The parameters of the DNN 104 may be updated iteratively for the number of epochs until the loss is a minimum (or below a threshold loss). In each epoch, the DNN 104 may be trained to separate the noise from the mixed signal 306 and output a denoised signal, which is the output signal. The output signal may be a cleaner version of the mixed signal, with the noise components removed or reduced. Details related to the training of the DNN 104 are further described, for example, in FIG. 3.

In some embodiments, the DNN 104 may learn to separate the noise from the mixed signal by being trained on a dataset where the first bio-signal serves as the ground truth, and the second bio-signal represents noise that is mixed with the first bio-signal. During the training process, the DNN 104 may be presented with the mixed signal, which is a composite of the ground truth bio-signal and the noise bio-signal. The training objective is to process the mixed signal in such a way that the DNN 104 may predict or reconstruct the ground truth bio-signal by filtering out the noise components from the mixed signal.

To achieve this, the DNN 104 may employ a variety of layers and transformations that enable it to learn complex patterns and dependencies within the mixed signal. As the DNN 104 processes the mixed signal through its layers, the DNN 104 gradually learns to distinguish between the characteristics of the ground truth bio-signal and the noise. This learning may be facilitated by the use of the spectral loss function that quantifies the difference between the DNN's output signal and the ground truth bio-signal. By minimizing this loss function during training, the DNN may effectively learn to suppress the noise while preserving the features of the ground truth bio-signal.

The training process involves adjusting the weights and biases of the DNN based on the computed loss, using optimization algorithms such as gradient descent. Over successive training epochs, the DNN 104 may become more adept at isolating and removing the noise components from the mixed signal, resulting in an output signal that closely resembles the ground truth bio-signal. This iterative process continues until the DNN 104 achieves a level of performance where the computed loss is below the threshold loss, indicating that the DNN 104 has effectively learned to denoise the mixed signal.

Based on the denoised output signal, the electronic device 102 may determine parameters related to a health condition of the user. Since the denoised output signal is cleaner than the input mixed signal, such parameters may be accurate. The disclosed electronic device 102 may thereby enable a robust and efficient determination of the health condition of the user and diagnosis of any adverse health issues related to the health condition. Furthermore, the electronic device 102 may provide a cost-effective solution for data augmentation with a wide coverage of the SNR to improve the performance of signal denoising. In some instances, the electronic device 102 may be incorporated in applications such as, intelligent medical and wearable devices to monitor health of patients and provide an early diagnosis of diseases, such as cardiovascular diseases (CVDs).

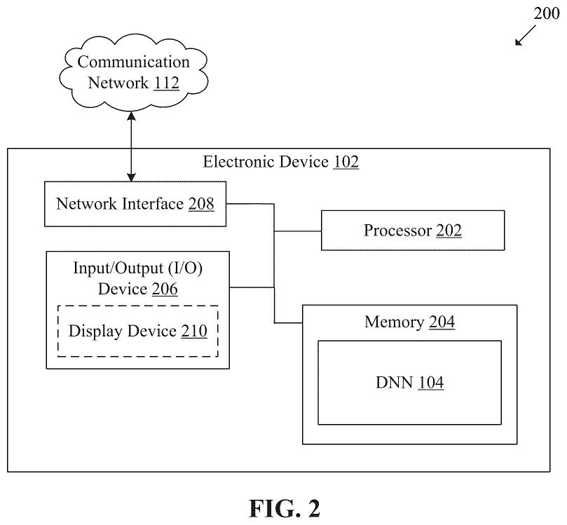

FIG. 2 is a block diagram that illustrates an exemplary electronic device of FIG. 1, in accordance with an embodiment of the disclosure. FIG. 2 is explained in conjunction with elements from FIG. 1. With reference to FIG. 2, there is shown the exemplary electronic device 102. The electronic device 102 may include a processor 202, a memory 204, an input/output (I/O) device 206, and a network interface 208. The memory 204 may store the DNN 104. The input/output (I/O) device 206 may include a display device 210.

The processor 202 may include suitable logic, circuitry, and/or interfaces that may be configured to execute program instructions associated with different operations to be executed by the electronic device 102. The operations may include, for example, a training data reception, a weighted sum computation, mixed signal generation, output signal generation, loss computation, and model training. The processor 202 may include one or more processing units, which may be implemented as a separate processor. In an embodiment, the one or more processing units may be implemented as an integrated processor or a cluster of processors that perform the functions of the one or more specialized processing units, collectively. The processor 202 may be implemented based on a number of processor technologies known in the art. Examples of implementations of the processor 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the processor 202. The one or more instructions stored in the memory 204 may be configured to execute the different operations of the processor 202 (and/or the electronic device 102). The memory 204 may be further configured to store the DNN 104, the training data for the DNN 104 (for example, the set of bio-signals 114 such as, the first bio-signal and the second bio-signal) or the bio-signal provided by the sensors 110. The memory may store the DNN 104 in a file format. The memory 204 may be a persistent memory, a non-persistent memory, or a combination thereof. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive the bio-signal associated with the user. The I/O device 206 may be further configured to display or render the denoised bio-signal. The I/O device 206 may include the display device 210. Examples of the I/O device 206 may include, but are not limited to, a display (e.g., a touch screen), a keyboard, a mouse, a joystick, a microphone, or a speaker. Examples of the I/O device 206 may further include braille I/O devices, such as, braille keyboards and braille readers.

The network interface 208 may include suitable logic, circuitry, interfaces, and/or code that may be configured to facilitate communication between the electronic device 102 and the server 106, via the communication network 112. The network interface 208 may be implemented by use of various known technologies to support wired or wireless communication of the electronic device 102 with the communication network 112. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 502.11a, IEEE 502.11b, IEEE 502.11g or IEEE 502.11n), voice over Internet Protocol (VoIP), light fidelity (Li-Fi), Worldwide Interoperability for Microwave Access (Wi-MAX), a protocol for email, instant messaging, and a Short Message Service (SMS).

The display device 210 may include suitable logic, circuitry, and interfaces that may be configured to display or render the denoised output signal associated with the user. The display device 210 may be a touch screen which may enable a user (e.g., the user 402 of FIG. 4) to provide a user-input via the display device 210. The touch screen may be at least one of a resistive touch screen, a capacitive touch screen, or a thermal touch screen. The display device 210 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 210 may refer to a display screen of a head mounted device (HMD), a smart-glass device, a see-through display, a projection-based display, an electro-chromic display, or a transparent display. Various operations of the processor 202 for implementation of adaptable deep neural networks for signal denoising are described further, for example, in FIG. 3.

FIG. 3 is a diagram that illustrates an exemplary training process of a denoising neural network, in accordance with an embodiment of the disclosure. FIG. 3 is explained in conjunction with elements from FIG. 1 and FIG. 2. With reference to FIG. 3, there is shown diagram 300 of an exemplary scenario for training process of the denoising neural network. In the exemplary scenario, training data 302 is provided as an input to compute a weighted sum 304 and to generate a mixed signal 306. The generated mixed signal 306 may be provided as an input to the DNN 308. Further, the DNN 308 may generate the output signal 310 based application of the DNN 308 on the mixed signal 306. Further, the processor 202 compares the output signal 310 with the first bio-signal 302A using the spectral loss function 312 to compute the loss 314. Furthermore, the processor 202 may be configured to train the DNN 308 based on the computed loss 314.

The training data 302 may be received by the processor 202. The training data 302 may include a first bio-signal 302A and a second bio-signal 302B. In some embodiments, the training data 302 may include more than two bio-signals. The second bio-signal 302B may be different from the first bio-signal 302A. Examples of the bio-signal may include, but are not limited to, EEG signal, ECG signal, EMG signal, EOG signal, an electroretinogram (ERG) signal, and an electrogastrogram (EGG) signal.

In an embodiment, the first bio-signal 302A may be the MCG signal, and the second bio-signal 302B may be the ECG signal. In another embodiment, the first bio-signal 302A may be the ECG signal, and the second bio-signal 302B may be the MCG signal. The MCG sensor may include a superconductive quantum interference device (SQUID) that may be positioned on chest of the user to measure the MCG signal. In order to obtain the ECG of the user, sensors such as the sensors 110 may be placed on skin of the user. In an example, ten electrodes may be placed on ten fingers and two electrodes may be placed on the chest of the user. Thereafter, an overall voltage measured from the twelve electrodes may be plotted against time in order to obtain the ECG of the user.

In another embodiment, the first bio-signal 302A may be the target signal, and the second bio-signal 302B may be the noise component or may include the noise component. The noise component may be a baseline wandering noise, a noise associated with human activities, an Electromyogram (EMG) noise, a channel noise, an electrode contact noise and the like.

The processor 202 may be configured to compute the weighted sum 304. The weighted sum 304 may be computed based on a first weight for the first bio-signal 302A and a second weight for the second bio-signal 302B. The second weight may be more or less than the first weight. In some embodiments, a noise signal may be pseudo randomly generated and used in the computation of the weighted sum. In such a case, the DNN 104 may learn to remove all signals in the weighted sum (or the mixed signal) other than the target signal. For example, the first bio-signal 302A may be the ECG signal, the second bio-signal 302B may be the MCG signal, and a third bio-signal may be a random noise signal. The ECG signal may be the target signal, and a combination of the random noise signal and the MCG signal may be noise components. Thus, the weighted sum 304 may be computed based on a first weight for the ECG signal, a second weight for the MCG signal and a third weight of the random noise signal. In an embodiment, the processor 202 may be configured to set each of the first weight and the second weight based on a user input or a random function. For example, the first weight may be greater than the second weight as per the user input.

The processor 202 may be configured to generate the mixed signal 306 based on the weighted sum 304. The mixed signal 306 may be generated through the process where each sample of the output signal is equal to the sum of weighted input (for example, a weighted input based on the first weight of the first bio-signal 302A and the second weight of the second bio-signal 302B).

The DNN 308 may be configured to generate an output signal based on application of the DNN 308 on the mixed signal 306. Herein, the DNN 308 may consist of two deep neural networks, i.e., the encoder 104A and the decoder 104B. In a first operation, the electronic device 102 may be configured to input mixed signal 306 to the encoder 104A to generate a latent signal as an output of the encoder 104A based on the application of the encoder 104A on the input mixed signal 306. As an example, the input mixed signal 306 may be generated based on the weighted sum of the first bio-signal 302A and the second bio-signal 302B (included in the received training data 302). Each bio-signal of the first bio-signal 302A and the second bio-signal 302B may include a set of channels that define the set of attributes for the corresponding signals.

In an embodiment, the DNN 308 may be a scalable deep-learning model comprising the encoder 104A, the decoder 104B. For the DNN 308 to be scalable deep-learning model, the electronic device 102 may include the DNN 308 with removed layers such that the DNN 308 may be scalable to various input size. The DNN 308 may take un-processed data such as, an unprocessed bio-signal to generate the output signal 310. The encoder 104A may receive the mixed signal 306 that may be based on the weighted sum 304 of the training data 302, as an input. Based on the received input, the encoder 104A may determine the compressed feature vector associated with the mixed signal 306. An encoded version (i.e., the compressed feature vector) of the received mixed signal 306 may be fed to the decoder 104B. The decoder 104B may learn to remove the second bio-signal 302B from the encoded version of the mixed signal 306 to provide the output signal 310 that includes the first bio-signal 302A. Alternatively, the decoder 104B may learn to remove the first bio-signal 302A from the encoded version of the mixed signal 306 to provide the output signal 310 that includes the second bio-signal 302B. Thus, the DNN 308 may be trained to separate the noise from the mixed signal and output a denoised signal (i.e., the output signal 310). The output signal 310 may be a cleaner version of the mixed signal, with the noise components removed or reduced.

In an embodiment, the DNN 308 may be a U-net or a variant of the U-net. The U-Net works as an encoder-decoder convolutional neural network designed for semantic segmentation tasks. The DNN 308 may be shaped like a letter U, with the encoder 104A, the decoder 104B, and skip connections coupling the encoder 104A with the decoder 104B. The encoder 104A may extracts more general features of the mixed signal 306 the deeper the encoder 104A encodes. The general feature may include features related to precise localization and context in the mixed signal 306. The decoder 104B may then use the extracted features to generate the output (such as the output signal 310). The skip connections may ensure that fine details (of the general features) are not lost during this process. The output of the DNN 308 may be a level map providing precise segmentation of the regions and layers of interest associated with each pixel in a biomedical image carried by the set of bio-signals in form of a dataset. In an embodiment, the layers of interest may be removed such that the DNN 308 is scalable to various input sizes. Further, the U-Net architecture may be trained in an end-to-end manner. In an embodiment, the DNN 308 may filter the noise component from the received training data 302 such that the output signal 310 is similar to the first bio-signal 302A.

The processor 202 may be configured to apply a spectral loss function 312 to the output signal 310. The spectral loss function 312 may be used to quantify the difference between the output signal 310 made by the DNN 308 and the target signal (for example, the first bio-signal 302A or the second bio-signal 302B). The spectral loss function 312 is the core of machine learning and deep learning, that are used to evaluate how well an algorithm is modelling the dataset.

The spectral loss function 312 may be used to minimize the loss 314, indicating that the DNN 308 predictions are getting closer to the accuracy. The processor 202 may be further configured to update the DNN 308 based on the estimated loss (for example, the loss 314) and output the updated encoder network as a trained DNN (for example a trained DNN 406 of FIG. 4) based on the estimated loss being a minimum.

In an embodiment the spectral loss function 312 may be based on a Short-Time Fourier Transform (STFT) or a Gabor transform. For example, the spectral loss function 312 may be based on STFT. The STFT is a Fourier-related transform (FT) that may be used to analyze the frequency and phase content of local sections of a signal (such as the first bio-signal 302A or the second bio-signal 302B or the mixed signal 306 or the output signal 310) over time. The STFT may divide the output signal 310 of the DNN 308 into shorter segments of equal length and computing the Fourier spectrum for each segment of the output signal 310. The STFT may be computed by multiplying the output signal 310 by a window function, sliding it along the time axis, and taking the FT of the output signal 310. The STFT may provide a two-dimensional representation of the signal in the time-frequency domain, allowing for the analysis of time-varying frequency components. In an embodiment, the STFT based spectral loss function 312 may be used in the context of training Variational Autoencoders (such as the DNN 308 of FIG. 3) to minimize loss generating samples. The spectral loss function 312 may emphasize local phase coherence between the first bio-signal 302A and output signal 310, focusing on the phase of high spatial frequencies to reduce perceived loss 314 of the first bio-signal 302A. Further, the STFT based spectral loss function 312 leverages the STFT to compute the loss between the first bio-signal 302A and output signal 310 based on local amplitudes and phases pertaining to higher frequencies.

The processor 202 may be further configured to train the DNN 308 based on the computed loss 314 and output the updated DNN 308 as the trained DNN (for example the trained DNN 406 of FIG. 4) based on whether the estimated loss is a minimum. By way of example, and not limitation, the training 316 of the DNN 308 may include an update of neural weights of the DNN 308 based on a back-propagation method. Additionally, or alternatively, the training 316 of the DNN 308 may include an update of one or more hyper-parameters of the DNN 308 before or after certain number epochs of the training 316 of the DNN 308 until the computed loss 314 is below a threshold loss. The updated DNN 308 or the trained DNN may output a more realistic denoised signal.

FIG. 4 is a diagram that illustrates an exemplary scenario of denoising the mixed signal 404, in accordance with an embodiment of the disclosure. FIG. 4 is described in conjunction with elements from FIG. 1, FIG. 2, and FIG. 3. With reference to FIG. 4, there is shown an exemplary scenario 400. The scenario 400 may include a user 402, the sensors 110, a mixed signal 404, a trained DNN 406, and the display device 210. The scenario 400 further illustrates a denoised bio-signal 408 (i.e., an output signal produced by the trained DNN 406). A set of operations associated the scenario 400 is described herein.

In the scenario 400 of FIG. 4, the sensors 110 may comprise a set of electrodes that may be positioned on limbs and chest of the user 402 to measure a bio-signal, such as an ECG signal or an MCG signal. Herein, the sensors 110 may be support a single channel input or a multi-channel input. In an embodiment, the bio-signal measured may include a noise component. The bio-signal with the noise component may be treated as a mixed signal 404.

At an inference stage, the trained DNN 406 may be configured to receive the mixed signal 404 via the single channel input or the multi-channel input of the sensors 110. Further, the trained DNN 406 may remove the noise component from the received mixed signal 404. The trained DNN 408 has learned to separate the noise component from the mixed signal 404 and output the denoised signal (such as the output signal 408), which may be, for example, a clean ECG or MCG signal. Upon removal of the noise component from the mixed signal 404, the DNN 406 may generate an output signal 408 (for example, a denoised bio-signal).

The processor 202 may render and display the generated denoised bio-signal on the display device 210. In certain embodiments, the display device 210 may display diagnosis parameters, such as heart rate and R-peak value based on the denoised bio-signal.

In an embodiment, the trained DNN 406 may be deployed for inference. The trained DNN 406 may include the decoder 104B. At the time of inference, the electronic device 102 may be configured to input mixed signal 404 to the decoder 104B and generate the output signal 408. For example, the mixed signal 404 may be a corrupted ECG signal. The ECG signal may be corrupted as the ECG signal may include the noise component. The mixed signal 404 is represented as graph of amplitude versus time as in FIG. 4. Since the ECG may be the electrical activity that may occur each time the heart of user 402 beats, the amplitude of the ECG signal may be a voltage. The voltage may be represented in milli-volts (mV) along a vertical axis of the ECG signal, and the time may be represented in seconds along a horizontal axis of the ECG signal.

At an inference stage, the trained DNN 406 may receive the corrupted ECG signal and may generate a denoised ECG signal by removing the noise component from the corrupted ECG signal. The denoised ECG signal may be rendered and displayed on the display device 210. In some instances, the denoised ECG signal of the user 402 may be displayed accurately for further diagnosis.

It should be noted that scenario 400 of FIG. 4 is for exemplary purposes and should not be construed to limit the scope of the disclosure.

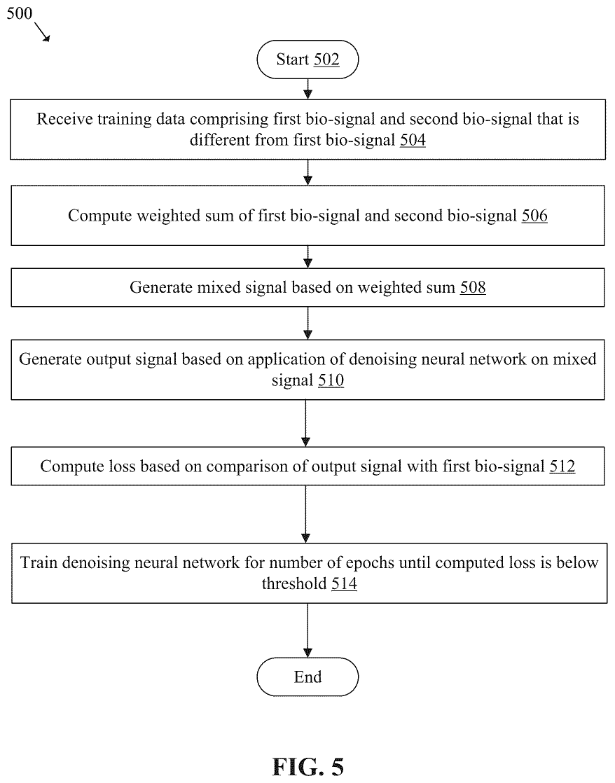

FIG. 5 is a flowchart that illustrates operations of an exemplary method for deep-learning based peak detection in bio-signal, in accordance with an embodiment of the disclosure. FIG. 5 is described in conjunction with elements from FIG. 1, FIG. 2, FIG. 3, and FIG. 4. With reference to FIG. 5, there is shown a flowchart 500. The flowchart 500 may include operations from 502 to 514 and may be implemented by the electronic device 102 of FIG. 1 or by the processor 202 of FIG. 2. The flowchart 500 may start at 502 and proceed to 504.

At 504, the training data 302 may be received. The processor 202 may be configured to receive the training data 302 comprising the first bio-signal 302A and the second bio-signal 302B that is different from the first bio-signal 302A. Details related to training data 302 are further described, for example, in FIG. 3 (at 302).

At 506, the weighted sum 304 of the first bio-signal 302A and the second bio-signal 302B may be computed. The processor 202 may be configured to compute the weighted sum 304 of the first bio-signal 302A and the second bio-signal 302B of the training data 302. Details related to the computation of the weighted sum 304 are further described, for example, in FIG. 3 (at 304 in FIG. 3).

At 508, the mixed signal 306 may be generated. The processor 202 may be configured to generate the mixed signal 306 based on the weighted sum 304. Details related to the mixed signal 306 are further provided, for example, in FIG. 3.

At 510, the output signal 310 may be generated. The processor 202 may be configured to generate the output signal 310 based on the application of the DNN 308 on the mixed signal 306. Details related to the output signals 310 are further described, for example, in FIG. 3 (at 308 and 310).

At 512, the loss 314 may be computed based on comparison of the output signal 310 and the first bio-signal 302A. The processor 202 may be configured to compute the loss 314 based on comparison of the output signal 310 with the first bio-signal 302A. Details related to the loss 314 are further described, for example, in FIG. 3 (at 314).

At 514, the DNN 308 may be trained (for example, the training 316 of FIG. 3). The processor 202 may be configured to train the DNN 308 for number of epochs until the computed loss 314 is below threshold. Details related to the training 316 of the DNN 308 are further described, for example, in FIG. 3 (at 316). Control may pass to end.

Although the flowchart 500 is illustrated as discrete operations, such as, 504, 506, 508, 510, 512, and 514 the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium having stored thereon, computer-executable instructions executable by a machine and/or a computer to operate an electronic device (for example, the electronic device 102 of FIG. 1). Such instructions may cause the electronic device 102 to perform operations that may include reception of a training data (for example, the training data 302 of FIG. 3) comprising a first bio-signal (for example, the first bio-signal 302A of FIG. 3) and a second bio-signal (for example, the second bio-signal 302B of FIG. 3) that may be different from the first bio-signal 302A. The operations may further include computation of a weighted sum (for example, the weighted sum 304 of FIG. 3) of the first bio-signal 302A and the second bio-signal 302B. The operations may further include generation of a mixed signal (for example, the mixed signal 306 of FIG. 3) based on the weighted sum 304. The operations may further include generation of an output signal (for example, the output signal 310 of FIG. 3) based on application of a denoising neural network (DNN) (for example, the DNN 308 of FIG. 3) on the mixed signal 306. The operations may further include computation of a loss (for example, the loss 314 of FIG. 3) based on a comparison of the output signal 310 with the first bio-signal 302A. The operations may further include training (for example, the training 316 of FIG. 3) of the DNN 308 for a number of epochs until the computed loss 314 is below a threshold.

Exemplary aspects of the disclosure may provide an electronic device (such as, the electronic device 102 of FIG. 1) that includes circuitry (such as, The processor 202). The processor 202 may be configured to receive training data (for example, the training data 302 of FIG. 3) comprising a first bio-signal (for example, the first bio-signal 302A of FIG. 3) and a second bio-signal (for example, the second bio-signal 302B of FIG. 3) that may be different from the first bio-signal 302A. The processor 202 may be configured to compute weighted sum (for example, the weighted sum 304 of FIG. 3) of the first bio-signal 302A and the second bio-signal 302B. The processor 202 may be configured to generate a mixed signal (for example, the mixed signal 306 of FIG. 3) based on the weighted sum 304. The processor 202 may be configured to generate an output signal (for example, the output signal 310 of FIG. 3) based on application of a denoising neural network (DNN) (for example, the DNN 308 of FIG. 3) on the mixed signal 306. The processor 202 may be configured to compute a loss (for example, the loss 314 of FIG. 3) based on a comparison of the output signal 310 with the first bio-signal 302A. The processor 202 may be configured to train (for example, the training 316 of FIG. 3) the DNN 308 for a number of epochs until the computed loss 314 is below a threshold.

In an embodiment, the first bio-signal 302A may be the MCG signal, and the second bio-signal 302B may be the ECG signal.

In an embodiment, the first bio-signal 302A may be one of the ECG signal, and the second bio-signal 302B may be the MCG signal.

In an embodiment, the loss 314 between the output signal 310 and the first bio-signal 302A is computed based on the spectral loss function 312.

In an embodiment, the spectral loss function 312 may be based on Short-Time Fourier Transform (STFT) or Gabor transform.

In an embodiment, the computation of the weighted sum 304 may be based on the first weight for the first bio-signal 302A and the second weight for the second bio-signal 302B, and the second weight is more than the first weight.

In an embodiment, the processor 202 may further configured to set each of the first weight and the second weight based on a user input or a random function.

In an embodiment, the processor 202 may be further configured to receive, from the sensor (for example, the sensors 110 of FIG. 1) associated with the user 402, the bio-signal comprising the noise component (for example, the mixed signal 404 of FIG. 4) via the single channel input or the multi-channel input, and generate the denoised bio-signal based on application of the trained DNN 406 on the received bio-signal, wherein the denoised bio-signal (for example, the output signal 408 of FIG. 4) is generated after the removal of the noise component from the received bio-signal.

In an embodiment, the DNN 104 may be the encoder-decoder network comprising the encoder 104A, the decoder block 104B coupled to the output of the encoder 104A.

In an embodiment, the DNN 104 may be the denoising autoencoder or the variant of the denoising autoencoder.

In an embodiment, the DNN may be the U-net or the variant of the U-net.

The present disclosure may also be positioned in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the embodiment disclosed, but that the present disclosure will include all embodiments that fall within the scope of the appended claims.

What is claimed is:

1. An electronic device, comprising:
a processor configured to:
receive training data comprising a first bio-signal and a second bio-signal that is different from the first bio-signal;
compute a weighted sum of the first bio-signal and the second bio-signal;
generate a mixed signal based on the weighted sum;
generate an output signal based on application of a denoising neural network (DNN) on the mixed signal;
compute a loss based on a comparison of the output signal with the first bio-signal; and
train the DNN for a number of epochs until the computed loss is below a threshold.

2. The electronic device according to claim 1, wherein the first bio-signal is a magneto cardiogram (MCG) signal, and the second bio-signal is an electrocardiogram (ECG) signal.

3. The electronic device according to claim 1, wherein the first bio-signal is one of an electrocardiogram (ECG) signal, and the second bio-signal is a magneto cardiogram (MCG) signal.

4. The electronic device according to claim 1, wherein the loss between the output signal and the first bio-signal is computed based on a spectral loss function.

5. The electronic device according to claim 4, wherein the spectral loss function is based on Short-Time Fourier Transform (STFT) or Gabor transform.

6. The electronic device according to claim 1, wherein the computation of the weighted sum is based on a first weight for the first bio-signal and a second weight for the second bio-signal, and the second weight is more than the first weight.

7. The electronic device according to claim 6, wherein the processor is further configured to set each of the first weight and the second weight based on a user input or a random function.

8. The electronic device according to claim 1, wherein the processor is further configured to:
receive, from a sensor associated with a user, a bio-signal comprising a noise component via a single channel input or a multi-channel input; and
generate a denoised bio-signal based on application of the trained denoising neural network on the received bio-signal,
wherein the denoised bio-signal is generated after a removal of the noise component from the received bio-signal.

9. The electronic device according to claim 1, wherein the DNN is an encoder-decoder network comprising an encoder, a decoder coupled to an output of the encoder.

10. The electronic device according to claim 1, wherein the DNN is a denoising autoencoder or a variant of the denoising autoencoder.

11. The electronic device according to claim 1, wherein the DNN is a U-net or a variant of the U-net.

12. A method, comprising:
in an electronic device:
receiving training data comprising a first bio-signal and a second bio-signal that is different from the first bio-signal;
computing a weighted sum of the first bio-signal and the second bio-signal;
generating a mixed signal based on the weighted sum;
generating an output signal based on application of a denoising neural network (DNN) on the mixed signal;
computing a loss based on a comparison of the output signal with the first bio-signal; and
training the DNN for a number of epochs until the computed loss is below a threshold.

13. The method according to claim 12, wherein the first bio-signal is a magneto cardiogram (MCG) signal, and the second bio-signal is an electrocardiogram (ECG) signal.

14. The method according to claim 12, wherein the first bio-signal is an electrocardiogram (ECG) signal, and the second bio-signal is a magneto cardiogram (MCG) signal.

15. The method according to claim 12, wherein the loss between the output signal and the first bio-signal is computed based on a spectral loss function.

16. The method according to claim 15, wherein the spectral loss function is based on Short-Time Fourier Transform (STFT) or Gabor transform.

17. The method according to claim 12, wherein the computation of the weighted sum is based on a first weight for the first bio-signal and a second weight for the second bio-signal, and the second weight is more than the first weight.

18. The method according to claim 17, further comprising setting each of the first weight and the second weight based on a user input or a random function.

19. The method according to claim 12, further comprising:
receiving, from a sensor associated with a user, a bio-signal comprising a noise component via a single channel input or a multi-channel input; and
generating a denoised bio-signal based on application of the trained denoising neural network on the received bio-signal,
wherein the denoised bio-signal is generated after a removal of the noise component from the received bio-signal.

20. A non-transitory computer-readable medium having stored thereon, computer-executable instructions that when executed by an electronic device, causes the electronic device to execute operations, the operations comprising:
receiving training data comprising a first bio-signal and a second bio-signal that is different from the first bio-signal;
computing a weighted sum of the first bio-signal and the second bio-signal;
generating a mixed signal based on the weighted sum;
generating an output signal based on application of a denoising neural network (DNN) on the mixed signal;
computing a loss based on a comparison of the output signal with the first bio-signal; and
training the DNN for a number of epochs until the computed loss is below a threshold.
